(19) United States
(12) Patent Application Publication
(10) Pub. No.: US 2004/0122357 A1
(43) Pub. Date: Jun. 24, 2004

(54) Use of a breast pump

(76) Inventors: Jacqueline Coral Kent, Nedlands (AU); Leon Robert Mitoulas, Osborne Park (AU); Donna Tracy Ramsay, Forrestfield (AU); Peter Edwin Hartmann, Gooseberry Hill (AU); Mark Derek Cregan, Scarborough (AU)

Correspondence Address: Michael H. Baniak, BANIAK PINE & GANNON, Suite 1200, 150 N. Wacker Drive, Chicago, IL, 60606, US

(21) Appl. No.: 10/401,138

(22) Filed: Mar. 27, 2003

(30) Foreign Application Priority Data
Dec. 20, 2002 (CH) 2002 2188/02

(51) Int. Cl. A61M 1/06

(52) U.S. Cl. 604/74

(57) ABSTRACT

A system for detecting a milk surge in a mother's breast has a breast pump (1) with a breast shield (10) for expressing milk from the breast, a collecting container (2), for receiving the milk expressed, and a unit (3, 4) by means of which a quantity of milk received in the collecting container (2) is determined as a function of time. This system makes it possible to detect the point in time and also the intensity of the milk surge. Application areas are research, in order to gain new knowledge about the breast-feeding behavior of babies and mothers, hospitals and child welfare clinics, in order to resolve breast-feeding problems, and development departments, for the purpose of optimizing breast pumps.

### TECHNICAL FIELD

The invention relates to a system and to a process for detecting a milk surge in a mother's breast and to the use of a breast pump for detecting a milk surge.

### PRIOR ART

The human breast has mammary glands which form balloon-like structures, so-called alveoli. The alveoli are connected to one another via milk ducts which lead to the nipple. The alveoli are enclosed by myoepithelial cells which contract under the influence of oxytocin. A sphincter at the end of the nipple, however, prevents the breast milk from flowing out.

If a baby then begins to suck at the breast, this is detected by mechanicoreceptors in the breast and a corresponding signal is transmitting to the mother's brain. Influenced by emotions, experiences and other external influences, the signal passes to the hypophysis which thus releases oxytocin.

The contraction of the myoepithelial cells triggered as a result leads to the alveoli deflating, as a result of which the milk ducts are widened. If the nipple sphincter is then opened by pressure, heat or other external factors, the milk can flow out. This contraction is referred to as the milk surge.

The prior art discloses breast pumps which are likewise able to express milk from the mother's breast. In particular WO 01/47577 discloses a milk pump which imitates the sucking rhythm of a baby and thus allows milk to be expressed from the breast in as natural a manner as possible.

Breast-feeding a baby, however, is not always straightforward. The causes of problems may stem from the baby or the mother or from both. It is often difficult, however, to determine the causes precisely. Expressing milk by means of a breast pump can also be problematic for some mothers. For the manufacturers of breast pumps, it is thus important to understand breast-feeding as precisely as possible and to utilize this knowledge in the design of breast pumps. It has been found that essential information for analyzing breast-feeding problems and for optimizing breast pumps may be derived from the mother's milk surge.

### DESCRIPTION OF THE INVENTION

It is an object of the invention to provide a system and a process which allows a milk surge to be detected in a straightforward manner.

This object is achieved by a system and a process having the features of patent claims 1 and 7, respectively.

In the process according to the invention, the milk is expressed into a collecting container and the quantity of milk expressed is determined as a function of time. If a milk surge takes place, then the quantity of milk detected increases abruptly. It is thus easy to detect the milk surge.

The quantity of milk is preferably weighed. However, other determining methods, for example volume measurements, are also possible.

The change in the quantity of milk over time is preferably directly determined or calculated. This allows the point in time and also the intensity of the milk surge to be detected to better effect.

The measurement results and measurement curves obtained in this way can be evaluated, and it is possible to draw conclusions about the behavior of the corresponding test individual in response to various external and internal influences. Application areas for the system and process according to the invention are, for example, research, in order to obtain knowledge about the breast-feeding behavior of babies and mothers. They may also be used, however, in hospitals or for advising mothers, in order to resolve breast-feeding or expressing problems. The results may also be used in product development, for the purpose of optimizing breast pumps.

Further advantageous variants and embodiments can be gathered from the dependent patent claims.

### BRIEF DESCRIPTION OF THE DRAWING

The subject matter of the invention is explained hereinbelow with reference to a preferred exemplary embodiment illustrated in the attached drawing, in which:

FIG. 1 shows a schematic illustration of the system according to the invention, and

FIG. 2 shows a measurement curve obtained by means of the process according to the invention and a calculated first derivative of the measurement curve.

### METHODS OF IMPLEMENTING THE INVENTION

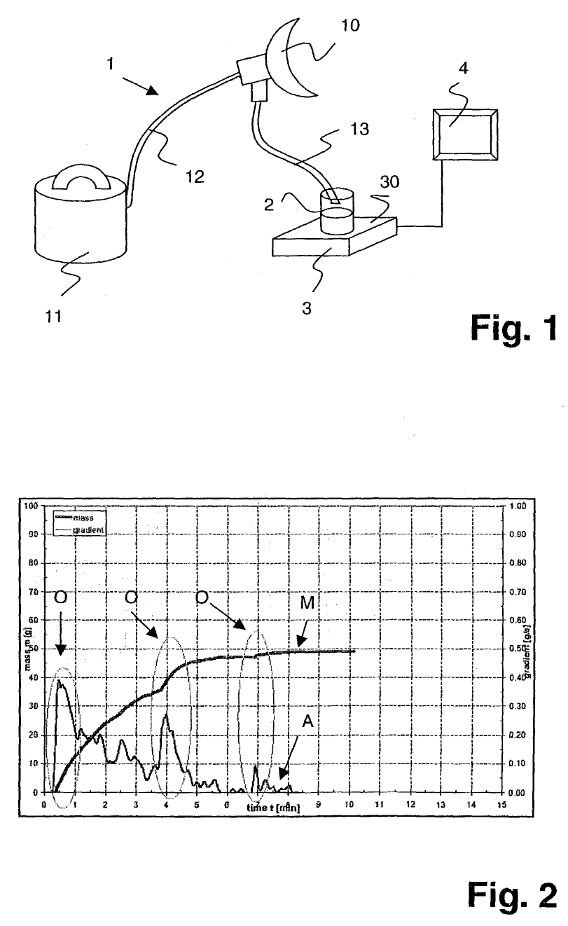

The system of the invention according to FIG. 1 has a breast pump 1, with at least one breast shield 10 for expressing the milk from a human breast, a collecting container 2, for receiving the milk expressed, and a unit with a measuring means 3 and an evaluating means 4, by means of which a quantity of milk received in the collecting container 2 is determined as a function of time.

The measuring means 3 serves for determining the weight or the change in weight of the breast milk located in the collecting container 2. In this preferred exemplary embodiment, this measuring means 3 is a balance, preferably an electromechanical balance with a bearing surface 30, on which the collecting container 2 is arranged.

The balance 3 is electronically connected to the evaluating means 4, which is preferably a computing system, in particular a computer, in order to transmit the measured values from the balance 3 to the computer 4. It is also possible, however, for the computer 4 and balance 3 to be integrated in a single machine. The measured values may be transmitted at defined time intervals or in a continuous manner.

In the evaluating means 4, the measured values and/or the change therein are illustrated as a function of time. FIG. 2 illustrates a corresponding measurement curve M and the derivative A thereof as a function of time. The y-axis shows the time in minutes, and the x-axis shows the mass in grams. The ovals O in FIG. 2 indicate the point in time of a milk surge. As can be seen, the flow of milk increases during a milk surge; the measurement curve M rises more rapidly. In the derivative A, the milk surges appear in the form of peaks and are thus even easier to make out. It can also be seen from the two curves M, A that the milk surge need not always have the same intensity. These curves can be evaluated and the results used for a variety of different purposes mentioned above.

FIG. 1 illustrates a table-top model of a breast pump. This means that the pumping unit is arranged in a housing 11 and is connected, by means of a negative-pressure tube 12, to the breast shield 10, in order for the negative pressure which is necessary for the flow of milk to be achieved therein. The breast shield 10 is connected to the collecting container 2 via a connecting tube 13, with the result that the breast milk can pass through this tube 13 into the container 2, for example a glass or a bottle. This apparatus has the advantage that the mother can move about during the test without falsifying the measurement result.

It is also possible, however, to use a breast pump in which the collecting container 2 is arranged on the breast shield 10. Here the important factor is for it to be possible to detect the behavior of the flow of milk over time.

List of designations

1 Breast pump

10 Breast shield

11 Housing

12 Negative-pressure tube

13 Connecting tube

2 Collecting container

3 Measuring means

30 Bearing surface

4 Evaluating means

M Measurement curve

A Derivative

O Oval

1. A system for detecting a milk surge in a mother's breast, the system having a breast pump with a breast shield for expressing milk from the breast, a collecting container, for receiving the milk expressed, and a unit by means of which a quantity of milk received in the collecting container is determined as a function of time.

2. The system as claimed in claim 1, wherein the unit has a measuring means, for measuring the quantity of milk located in the collecting container, and an evaluating means by means of which the quantity of milk measured is evaluated as a function of time.

3. The system as claimed in claim 2, wherein the measuring means is a balance.

4. The system as claimed in claim 3, wherein the balance is an electromechanical balance with a bearing surface for the collecting container.

5. The system as claimed in claim 2, wherein the evaluating means is a computing system, in particular a computer.

6. The system as claimed in claim 1, wherein the collecting container is connected to the breast shield via a connecting tube.

7. A process for detecting a milk surge in a mother's breast, milk being expressed from the breast into a collecting container, and the quantity of milk expressed being determined as a function of time.

8. The process as claimed in claim 7, wherein the weight of the quantity of milk expressed is determined as a function of time.

9. The process as claimed in claim 7, wherein the change in weight of the quantity of milk expressed is determined as a function of time.

10. The process as claimed in claim 7, wherein, in order to express the milk, use is made of a breast pump with a breast shield, and wherein the milk expressed is directed from the breast shield into the collecting container via a connecting tube.

11. Use of a breast pump for detecting a milk surge in a mother's breast, milk being expressed from the breast into a collecting container by means of the breast pump, and the quantity of milk expressed being determined as a function of time.
